(19) United States
(12) Patent Application Publication
(10) Pub. No.: US 2004/0198841 A1
(43) Pub. Date: Oct. 7, 2004

(54) Use of thiol-based compositions in ameliorating mucosal injury

(75) Inventor: Edward A. Neuwelt, Portland, OR (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Oregon Health & Science University, Portland, OR

(21) Appl. No.: 10/801,122

(22) Filed: Mar. 15, 2004

Related U.S. Application Data
(60) Provisional application No. 60/454,886, filed on Mar. 13, 2003.

(51) Int. Cl. A61K 31/10

(52) U.S. Cl. 514/711

(57) ABSTRACT

The present invention discloses a method of ameliorating mucosal injury in patients undergoing chemotherapy by the administration of a thiol-based compound or composition.

### CROSS-REFERENCE TO RELATED APPLICATION

This application claims the benefit of U.S. Provisional Patent Application No. 60/454,886, filed Mar. 13, 2003, which is incorporated herein by reference in its entirety.

### BACKGROUND OF THE INVENTION

1. Field of the Invention

The present invention is directed to a method of preventing and treating mucosal injury. In particular, the invention is directed to a method of preventing and treating mucosal injury by the administration of a thiol-based scavenger, such as N-acetylcysteine (NAC), or sodium thiosulfate.

2. Description of the Related Art

Patients undergoing chemotherapy and radiation therapy to treat cancer frequently suffer from mucosal injury. Mucosal injury is often painful and interferes with completing a course of treatment. Mucosal injury involves damage to the mucosal lining of the mouth, gastrointestinal tract, and any cavity lined by a mucous membrane. Membranes lining passages and cavities communicating with the air can be considered mucous membranes (Taber's Cyclopedic Medical Dictionary, Edition 17, 1993). Mucous membranes consist of at least a surface layer of epithelium. Epithelial cells are derived from either fibroblasts or other transitional cells. Mucous secreting cells or glands are usually present in the epithelium, but may be absent. Without being bound to a mechanism, the mechanism of chemotherapy-induced mucositis appears to arise from a combination of many factors. Presumably, chemotherapy damages the rapidly dividing immature intestinal crypt cells and more superficial immature mucosal cells in the oropharynx. In addition to this direct damage, it is theorized that, as the mature epithelial cells are sloughed, damaged immature cells are exposed to pancreatic and biliary secretions resulting in further intestinal damage. This damage contributes to mucositis. Mucosal injury presents itself clinically in many forms, including, but not limited to, mucositis, xerostomia, esophagitis, upper gastrointestinal bleeding, osteoradionecrosis (ORN), and colitis.

The impact of radiotherapy on the mouth primarily results in local tissue changes and includes both acute and latent effects. Consequently, the dose rate and total dose of radiotherapy to the oral cavity directly relate to the extent and type of injury. Oral tissues that are affected directly by radiation include the epithelium, salivary glands, bone, and muscle. The teeth may be secondarily affected as a consequence of radiation-induced xerostomia (abnormal dryness of the mouth).

Mucositis

Direct epithelial injury is an important component of radiation-induced mucositis, however, it appears to be only one segment of a cascade which includes free radical formation, endothelial and connective injury, fibronectin degeneration, and proinflammatory cytokine release and expression. While the changes in epithelial proliferation are noted at a level of 20 Gy when therapy is administered at a rate of 200 cGy daily, it appears the sequence leading to mucosal injury begins earlier. Clinically, erythema and edema characterize the early changes of mucositis and generally begin about two weeks after the start of therapy. The movable mucosa of the cheeks, lips, soft palate, and ventral surface of the tongue most often are affected. As the cumulative dose of radiation increases, so too does the advancement from erythema to ulceration. Because trauma accelerates the progression to ulcer formation, treatment has consisted of eliminating sources of local irritation before the initiation of radiotherapy.

The ulcerative lesions of mucositis are markedly symptomatic. The resulting pain may be of such severity as to necessitate the use of parenteral analgesics or the interruption of radiotherapy. Lesions tend to be self-limiting and usually disappear after 2 to 3 weeks following the completion of radiation therapy.

The severity of radiation-induced mucositis relates to the dose rate and total dose of therapy as well as to the presence of local irritation, secondary infection, and xerostomia. Pretreatment elimination of local sources of irritation is an important aspect of preventing mucositis. The various pretreatment to date have included benzydamine hydrochloride (HCl), a nonsteroidal rinse with anti-inflammatory, analgesic, and anesthetic properties; sucralfate, which is an agent that has received wide use in the treatment of gastric ulcers and forms a protein/drug complex on the site of ulcerated mucosa; lidocaine (Xylocalne), dyclonine HCl (Dyclone), and benzocaine in orabase which are a variety of topical palliative agents which exist to manage the pain and sensitivity that are associated with mucositis; diphenhydramine HCl (Benadryl) has topical anesthetic activity, may be mixed as a suspension with equal parts of either Kaopectate or Milk of Magnesia; chlorohexidine gluconate, lozenges containing polymyxine E, tobramycin and amphotericin have been used to prevent microbial colonization of disrupted mucosa; anti-inflammatory agents such as betamethasone and indomethacin; and immunoglobulin therapy, which appears to be of value in the treatment of other mucosal diseases, has been the subject of a hematologic cytokine therapy, particularly with granulocyte-macrophage colony-stimulating factor (GM-CSF), as a treatment for mucositis, on the basis of the rationale that such treatment might favorably effect the course of mucositis by affecting the local immune response. Amifostine has also been administered to patients undergoing radiation therapy in an attempt to treat or prevent mucositis, but was found not to reduce mucositis. Brizel DM, J Clin Oncol 1;18(19):3339-45 (October 2000).

Xerostomia

Xerostomia is one of the most consistent and bothersome side effects of radiation therapy, and it may be exacerbated by concomitant chemotherapy. Xerostomia is caused by the effects of radiation on acinar cells, especially of the serous glands (i.e., parotid glands). Consequently, inflammation, degeneration, and fibrosis of the glandular parenchyma occur. The extent, duration, and degree of recovery are a function of the dose rate, total dose, and radiation port. Onset of xerostomia may be noted as early as one week following the onset of radiation in which the salivary glands, especially the parotid, are exposed. The saliva turns thick and ropey as serous function is diminished. Patients who receive radiation to the head and neck in cumulative doses of 60 Gy or more usually have irreversible xerostomia, with an 80% loss in salivary gland function. Spontaneous recovery is unlikely for patients with xerostomia persisting for 12 months or longer; with lesser doses, however, the inflammation and edema of glandular tissue often disappear spontaneously within 12 months after therapy.

In addition to functional changes caused by xerostomia, such as difficulty in swallowing and alteration of taste, loss of saliva also is associated with a reduction in oral flushing and diminished oral immunoglobulin A (IgA) levels and salivary antibacterial enzymes. Consequently, patients with xerostomia are susceptible to increases in local oral infections, including caries, periodontal disease, and candidiasis.

Radiation-induced caries can be a common problem in patients with xerostomia. Changes in salivary composition, decreases in buffering capacity, and loss of the cleansing action of saliva result in the accumulation of bacteria, increases in the local oral cariogenic flora, and tooth decalcification with consequent caries development. Typically, radiation caries present with lesions at the cervical margins of the teeth, which then progress rapidly. Decalcification of the incisal edges of the teeth also may be noted. In addition to tooth loss, a major consequence of uncontrolled caries may be abscess formation in patients who are at risk for osteoradionecrosis.

The radioprotective agent WR-2721 (amifostine), a free-radical scavenger, has been approved for use in the prevention of radiation-induced xerostomia. Wasserman, T., Semin Oncol 26 (2 Suppl 7):89-94, April 1999. The recommended dose for amifostine is 200 mg/m² administered once daily as a 3-minute intravenous infusion, starting 15 to 30 minutes prior to standard fraction radiation therapy. The need for intravenous infusion, the frequency of dosing, and the potential side effects associated with amifostine will likely affect its frequency of use.

Loss of taste is a transient but bothersome sequela of head and neck radiation. The severity of taste loss increases rapidly up to doses of 30 Gy but then plateaus. Patients receiving doses of 30 Gy or more may lose their ability to distinguish salt or sweet tastes. Fortunately, hypogeusia for most patients is transient, and taste begins to return within 1 or 2 months after therapy. Total recovery, however, may take up to 12 months.

Of all the oral complications of head and neck radiation, the most significant is ORN. First described in 1922, ORN results in the denudation of soft tissue and both exposure and necrosis of bone. Although not limited to the jaws, it frequently is found in this location. ORN results in a painful, chronic, open, and foul-smelling wound that is of great distress to the patient. Most cases heal with conservative therapy, but the course usually is prolonged. Historically, ORN was attributed to the triad of trauma (often tooth extraction), radiation, and infection. Subsequent studies, however, suggest that ORN represents a defect of wound healing rather than a true osteomyelitis. The etiology appears to relate to diminished vascularization as a consequence of radiotherapy. Histologic changes of thickened arterial and arteriolar walls substantiate this hypothesis, and the lack of culturable pathogenic microorganisms from active ORN lesions suggests a noninfectious nature of the process.

No consensus exists concerning the overall frequency of ORN. Although reported ranges vary between 4 and 44%, approximately 15% appears to be the preponderant experience. The mandible more often is involved than the maxilla, which probably reflects the difference in blood supply and vascularity of the two bones. Time until onset of ORN following radiotherapy is controversial. Some authors have described ORN as early as 2 weeks after radiotherapy, while others report it as a late condition. Most cases occur within the first year after radiotherapy.

Equally controversial is the rate at which the risk of ORN diminishes with time after the completion of radiotherapy, although it seems clear that ORN can occur at any time after radiotherapy.

The field size, dose rate, and total dose of radiotherapy have a marked effect on the frequency of ORN. Patients who receive cumulative doses of 65 Gy or more to the mandible or maxilla are more likely than patients receiving lesser doses to develop ORN. For example, patients who receive 80 Gy or more are twice as likely as patients who receive between 50 to 60 Gy to develop ORN. Patients with tumors that are adjacent or contiguous to the bone also are at a higher risk of developing ORN. This finding likely is the result of the inclusion of bone in the radiated field because the volume of bone that is exposed to radiotherapy has a direct effect on ORN. Poor nutrition and immune status also appear to predispose to the condition.

Esophagitis

In addition to oral mucositis, esophageal injury and infection are frequently recognized in patients undergoing cancer treatment and in the immunocompromised host. Esophagitis can be caused by cytotoxic chemotherapy and irradiation as well as by viral, bacterial, and fungal organisms. Radiation esophagitis commonly occurs during treatment of intrathoracic malignancies, particularly lung and esophageal cancers. The frequency and severity of esophagitis increases with radiation dose and with the use of certain chemotherapeutic agents, including doxorubicin, bleomycin, cyclophosphamide, and cisplatin. Symptoms include odynophagia and dysphagia as well as retrosternal chest pain. At endoscopy, findings include erythema, edema, and friability of the esophageal mucosa, as well as ulceration with eventual stricture formation. Strictures result from submucosal fibrosis and degenerative changes involving blood vessels.

Symptomatic strictures can be managed with esophageal dilation. Current treatments include relief of odynophagia with viscous lidocaine during the acute phase and use of H₂-blockers or proton pump inhibitors to prevent further acid-related injury.

Upper Gastrointestinal Bleeding

In patients receiving chemotherapy, retching and nausea/vomiting can be controlled with antiemetics, including serotonin antagonists. However, emetogenic injury to the gastric mucosa and the gastroesophageal junction (Mallory-Weiss tear) commonly occur and produce upper gastrointestinal bleeding. These injuries can produce very significant bleeding in the setting of thrombocytopenia. The etiology of upper gastrointestinal bleeding in patients with cancer is commonly due to benign causes. The development of thrombocytopenia and/or coagulopathy can unmask focal pathology and lead to gastrointestinal bleeding. Patients with cancer and that undergoing cancer treatment are at risk for stress-related mucosal injury.

Stress-related mucosal injury is a common problem frequently seen in critically ill patients, including those with cancer. Many terms have been associated with this entity, including stress-related mucosal damage, stress ulceration, erosive gastritis, and stress ulcer syndrome. Painless, occult or overt upper gastrointestinal bleeding can develop in up to 20% of patients in an intensive care unit (ICU) setting. Significant hemorrhage is reported to occur in approximately 6% of patients. The likelihood of significant bleeding from stress-related mucosal lesions depends upon risk factors such as thrombocytopenia, coagulopathy, sepsis, major surgical procedures, and the presence of organ failure. Use of nonsteroidal anti-inflammatory drugs (NSAIDs) is also a risk factor. Endoscopic findings include multiple superficial erosions or ulcers that arise most often in the gastric fundus.

Pseudomembranous Colitis

Clostridium difficile is the most common bacterial cause of infectious diarrhea in antibiotic-treated patients and in those undergoing cancer chemotherapy. Essentially any antibiotic can cause this syndrome, however, those drugs that are prescribed most frequently (i.e., cephalosporins followed by the penicillins) are most commonly implicated. Cancer patients receiving chemotherapy appear predisposed to C. difficile-induced diarrhea even in the absence of antibiotics. In a study of such patients, methotrexate, doxorubicin, and cyclophosphamide were the drugs most frequently associated with C. difficile infection. It is speculated that anticancer-drug-mediated mucosal injury may produce the anaerobic environment conducive to C. difficile colonization.

Diarrhea is the key feature and is usually watery, voluminous, and without gross blood. Most patients have abdominal pain and tenderness, fever, and leukocytosis, although symptoms vary and generally begin after 5 to 10 days of antibiotic therapy; however, they may occur as late as 3 to 4 weeks after discontinuation of therapy.

The treatment of antibiotic-associated pseudomembranous colitis requires discontinuation of the implicated antibiotic. Many patients improve spontaneously with only this measure; however, specific therapy shortens the duration of symptoms. The most widely used agent is oral vancomycin, which like metronidazole, is poorly absorbed and reaches high concentrations in the stool.

Further, as disclosed in U.S. Pat. No. 6,416,955, incorporated herein in its entirety, between 20 to 40 million Americans suffer from chronic rhinosinusitis, an inflammation of the nasal cavity and/or paranasal sinuses. In addition, chronic rhinosinusitis has been estimated to account for up to 90 percent of all cases of rhinosinusitis with acute rhinosinusitis (e.g., allergic rhinitis) accounting for the remaining 10 percent. While it is known that large numbers of eosinophils infiltrate the nasal tissue in patients with chronic rhinosinusitis as well as in patients with allergic rhinitis, the pathophysiology of these and other mucositis conditions remains unknown.

There is a need for an anti-mucosal injury agent which is more effective than current agents to prevent or treat mucosal injury in persons undergoing chemotherapy and which correspondingly does not protect the tumor from the chemotherapeutic agent. The present invention meets this need and provides other related advantages.

### BRIEF SUMMARY OF THE INVENTION

The present invention provides methods for preventing or ameliorating chemotherapeutic agent-induced mucosal injury and its associated symptoms (e.g., cachexia). Such methods comprise administering to a patient in need thereof an effective amount of a thiol-based compound or composition.

The thiol-based compounds of the present invention may be administered intravenously, intra-arterially, intra-peritoneally, orally, intradermally, subcutaneously, transdermally, nasally, or anally. In certain embodiments, the thiol-based compound is administered orally, intravenously or intra-arterially.

In certain embodiments, the thiol-based compound is administered prior to the administration of the chemotherapeutic agent or at least one of the chemotherapeutic agents. In other embodiments, the thiol-based compound is administered concurrently with the administration of the chemotherapeutic agent or at least one of the chemotherapeutic agents. In certain embodiments, the thiol-based compound is administered following the initiation or the completion of the administration of the chemotherapeutic agent or at least one of the chemotherapeutic agents. For instance, the thiol-based compound may be administered at least or about 15 minutes, 30 minutes, 45 minutes, 1 hour, 2 hours, 3 hours or 4 hours prior to the beginning of the administration of the chemotherapeutic agent or agents.

The thiol-based compound or composition may be selected from a group consisting of sodium thiosulfate, N-acetyl cysteine, glutathione ethyl ester, D-methionine, S-adenosyl-methionine, cysteine, N,N′-diacetyl-cysterine, cystathione, glutathione, glutathione ethyl ester, glutathione diethyl ester, S-(1,2-dicarboxyethyl) glutathione triester, cysteamine, cysteine isopropylester, and combinations thereof. In certain embodiments, the thiol-based compound or composition is sodium thiosulfate (STS), N-acetyl cysteine (NAC), or combinations thereof.

The chemotherapeutic agent may be any compound that is administered to a mammalian subject to destroy, or otherwise adversely affect, cancer cells. Such compounds may be platinum derivatives, taxanes, steoid derivatives, anti-metabolites, plant alkaloids, antibiotics, arsenic derivatives, intercalating agents, alkylating agents, enzymes, biological response modifiers and combinations thereof. In certain embodiments, the chemotherapeutic agents are alkylating agents, such as platinum-containing alkylating agents. Exemplary platinum-containing alkylating agent may be cisplatin, carboplatin, oxyplatin, or combinations thereof. In certain embodiments, the chemotherapeutic agent or one of the chemotherapeutic agents is carboplatin, cisplatin, or BR96-dox.

A patient in need of prevention or amelioration of chemotherapeutic agent-induced mucosal injury may be a human, a non-human primate, or another mammal that will undergo (or is undergoing) chemotherapy and is at high risk of (or is suffering from) a chemotherapeutic agent-induced mucosal injury. In certain embodiments, the patient may suffer from tumor in the head or neck (e.g., brain tumor or cancer). In other embodiments, the patient may suffer from tumor or cancer located otherthan head or neck. In certain embodiments, the patient receives a blood brain barrier disruption procedure. In other embodiments, the patient does not receive a blood brain barrier disruption procedure.

The dosage of using N-acetylcysteine, an exemplary thiol-based compound, in preventing or ameliorating mucosal injury may be at least or about 150, 200, 300, 400, 500, 600, 700, 800, 900, 1000, 1100, 1200, 1300 or 1400 mg/kg in humans. In addition, multiple doses (e.g., 2, 3, 4, 5, 6, 8, or 10) may be used.

### BRIEF DESCRIPTION OF THE SEVERAL VIEWS OF THE DRAWINGS

FIG. 1 shows a graph representing the weight loss in grams in Long Evans rats seven days after administration of cisplatin with and without NAC (400 mg/kg) given intravenously prior to cisplatin, which causes renal damage and diarrhea.

FIG. 2 shows a graph representing weight loss in grams in nude rats 12 days after intracerebral inoculation with SCLC cells, with and without treatment with Temazolamide given orally once daily on days 6-11.

FIG. 3 shows cytoenhancement and chemoprotection in fibroblasts cells, the type of cells that line the mouth and esophagus. Cytotoxicity was assessed in GM294 human fibroblasts, 1×10⁴ cells per well in 96 well plates, using the WST colorometric assay. Cells were pretreated with or without BSO, 100 μM for 18 hours prior to addition of chemotherapeutics (melphalan=10 μg/ml, carboplatin=100 μg/ml, cisplatin=7.5 μg/ml, etoposide phosphate 100 μg/ml) either alone (open bar), or with NAC rescue, (1000 μg/ml N-acetylcysteine, striped bar), BSO cytoenhancement (black bar), or BSO cytoenhancement and NAC rescue (cross hatched bar). Data are expressed as the percentage of live cells compared to control samples (without chemotherapy) and each point represents the mean±s.d. of 4 wells. This was also repeated with normal human gastric cells (NHGC) and gastric epithelial cells.

FIG. 4 shows a graph representing the results, in NHGC, of a WST-1 assay of the chemoprotective effect of NAC on BR96-dox and Carboplatin.

FIG. 5 shows a graph representing NAC treated rats which exhibited less overall toxicityto cisplatin (CDDP) as evidenced by lower weight loss 7 days post treatment in rats treated with NAC.

### DETAILED DESCRIPTION OF THE INVENTION

The present invention provides methods for preventing or reducing chemotherapeutic agent-induced mucosal injury while not substantially affecting efficacy of the chemotherapeutic. Such methods comprise administering to a patient in need thereof an effective amount of a thiol-based compound or composition, such as sodium thiosulfate and N-acetylcysteine. In certain embodiments, the thiol-based compound (or composition) and the chemotherapeutic agent are administered separately to avoid interference of the thiol-based compound (or composition) on anti-tumor efficacy of the chemothepeutic agent(s). Such separation may be temporal, spatial, or both.

As one of skill in the art can readily appreciate, the ability to prevent or reduce mucosal injury is only relevant if the chemotherapeutic drug retains efficacy toward the tumor. The present invention meets this rigorous standard and thus provides a unique approach to preventing or reducing mucosal injury.

As used therein, “mucosal injury” refers to damage to the mucosal lining of the mouth, gastrointestinal tract and any cavity lined by mucous membrane. Mucosal injury presents itself clinically in many forms, including, but not limited to mucositis, xerostomia, esophagitis, upper gastrointestinal bleeding, osteoradionecrosis and colitis.

“Chemotherapeutic agent” refers to a compound that is administered to a mammalian subject to destroy, or otherwise adversely affect, cancer cells. Chemotherapeutic agents include, but are not limited to, platinum derivatives (e.g., cisplatinum and carboplatinum), taxanes (e.g., paclitaxel), steroid derivatives, anti-metabolites (e.g., 5-fluorouracil, methotrexate and cytosine arabinoside), plant alkaloids (e.g., vindesine VP16, vincristine and vinblastine), antibiotics (e.g., adriamycin, mitomycin C, bleomycin, mithramycin, daunorubicin, mitoxantrone, and doxorubicin), etoposide, arsenic derivatives, intercalating agents, alkylating agents (e.g., melphalan, cyclophosphamide, chlorambucil, busulphan, thiotepa, isofamide, mustine, and the nitrosoureas), enzymes (e.g., asparaginase), biological response modifiers (e.g., immunoadjuvants and immunorestoratives), hydroxyurea, procarbazine, and combination thereof. In certain embodiments, chemotherapeutic agents are alkylating agents. In certain embodiments, alkylating agents are platinum-containing alkylating agents (e.g., cisplatin, carboplatin, and oxyplatin).

“Chemotherapeutic agent-induced mucosal injury” (interchangeably used with “chemotherapy-induced mucosal injury”) refers to mucosal injury caused or induced by the administration of a chemotherapeutic agent or a combination of chemotherapeutic agents.

“Preventing a chemotherapeutic agent-induced mucosal injury” refers to preventing or diminishing the occurrence of chemotherapeutic agent-induced mucosal injury. A subject in need of prevention of chemotherapeutic agent-induced mucosal injury refers to a human, non-human primate or other mammal that will undergo, or is undergoing, chemotherapy and is at high risk for chemotherapy-induced mucosal injury.

A subject at risk for chemotherapy-induced mucosal injury is one that has at least one of the risk factors for chemotherapy-induced mucosal injury. For instance, patients receiving minimally myelosuppressive or nonmyelosuppressive chemotherapy are at lower risk for oral musocal injury. Patients receiving stomatotoxic chemotherapy resulting in prolonged myelosuppression (including those undergoing blood and marrow transplantation) and patients undergoing head and neck radiation for oral, pharyngeal, and laryngeal cancer are at higher risk for oral musocal injury.

“Ameliorating chemotherapeutic agent-induced mucosal injury” refers to reducing the severity of chemotherapeutic agent-induced mucosal injury. A subject in need of ameliorating a chemotherapeutic agent-induced mucosal injury refers to a human, non-human primate or other animal that is undergoing chemotherapy and suffers from a chemotherapeutic agent-induced mucosal injury.

“Thiol-based compound” refers to a compound containing a thio, thiol, aminothiol or thioester moiety. Thiol-based compounds include, but are not restricted to, sodium thiosulfate, N-acetyl cysteine, glutathione ethyl ester, D-methionine, S-adenosyl-methionine, cysteine, N,N′-diacetyl-cysterine, cystathione, glutathione, glutathione ethyl ester, glutathione diethyl ester, S-(1,2-dicarboxyethyl) glutathione triester, cysteamine, and cysteine isopropylester. However, in the context of the present invention, thiol-based compound does not include thiol amifostine (Ethyol or WR 2721). If a thiol-based compound contains one or more amino acid residues, the amino acid residues may be in a L- or D-form. Thiol-based compound of the present invention may be used individually or in combination with one or more other thiol-based compounds, and/or other pharmaceutical agents and excipients.

“Thiol-based composition” refers to a composition comprising at least one thiol-based compound. Such compositions may also include, in addition to one or more thiol-based compounds, pharmaceutically acceptable carriers that facilitate administration of thiol-based compound(s) to a mammalian subject.

The term “effective amount” refers to an amount of thiol-based compound or composition that is sufficient to prevent or reduce chemotherapeutic agent-induced mucosal injury.

The present application provides thiol-based compositions and methods for using such compositions in preventing or ameliorating chemotherapy-induced mucosal injury. Techniques for the formulation and administration of the compounds of the present application may be found in “Remington's Pharmaceutical Sciences” Mack Publishing Co., Easton, Pa., latest edition.

The thiol-based compounds of the present invention are formulated to be compatible with their intended route of administration. Examples of route of administration include intravenous (i.v.), intra-arterial (i.a.), intra-peritoneal (i.p.), oral (p.o.), intradermal, subcutaneous, transdermal, intranasal, and intra-anal administration. Solutions or suspensions used for intravenous, intra-arterial, intradermal, or subcutaneous application can include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In addition, pH may be adjusted with acids or bases, such as hydrochloric acid or sodium hydroxide. The thiol-based compounds are preferably administered in their un-oxidized form. The parenteral preparation can be enclosed in ampoules, disposable syringes or multiple dose vials made of glass or plastic.

Thiol-based compounds suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against contamination from microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In certain embodiments, a spatial two-compartment pharmacokinetic model is used to administrating NAC (and/or other thiol-based compounds) and chemotherapeutic agent(s). Any models known in the art that is suitable for spatially separating the chemotherapeutic agent(s) from chemoprotectants (e.g., NAC and other thiol-based compounds) may be used. Such separation allows for the reduction of chemotherapy-induced toxicity without affecting chemotherapy efficacy. Exemplary two-compartment pharmacokinetic models may be found in published PCT Application No. WO 01/80832. For instance, head and neck tumors are treatable through regionalization of chemotherapeutic agents to head and neck where the tumor tissue is located and through regionalization of chemoprotectants (e.g., NAC) to general tissues below the level of the heart where the majority of bone marrow tissue is located. An example of spatial compartmentalization is the administration of a chemoprotectant into the descending aorta or lower, preventing any significant chemoprotectant concentrations of the protectant from ever reaching head or neck where the tumor tissue is located.

In certain embodiments, thiol-based compounds (e.g., sodium thiosulfate) are administered i.v. This route of administration is especially useful in preventing or ameliorating mucosal injury induced by chemotherapy for treating head or neck tumor and brain cancer. Intravenous administration of thiol-based compounds (e.g., sodium thiosulfate and N-acetylcysteine) results in minimum amount of thiol-based compounds in brain due to the blood brain barrier, which in turn prevents or reduces neurotoxicity of these thiol-based compounds and adverse effects of these compounds on chemotherapy efficiency.

It is advantageous to formulate compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Various animal models and clinical assays for evaluating effectiveness of a particular thiol-based compound in preventing or reducing bone marrow known in the art may be used in the present invention. They include, but are not limited to, those described in Clarkson et al., Cochrane Database Syst. Rev. 2003; (3): CD000978; Barasch and Peterson et al., Oral Oncol. 39: 91-100,2003; Okajima et al., Crit. Care Med. 28: 2858-65, 2000; Fiorucci et al., Aliment Pharmacol. Ther. 12:1139-53,1998; and Salim, Lancet 2(8676): 1390, 1989. Additional assays are described in the examples below.

The dosage of using N-acetylcysteine in preventing or ameliorating mucosal injury, when administered intravenously, may be at least or about 150, 200, 300, 400, 500, 600, 700, 800, 900, 1000, 1100, 1200, 1300, or 1400 mg/kg in humans, or a dosage in another subject comparable to that in humans. A dosage (“dosage X”) of a thiol-based compound in a subject other than a human is comparable (or equivalent) to a dosage (“dosage Y”) of the thiol-based compound in humans if the serum concentration of the scavenger in the subject post administration of the compound at dosage X is equal to the serum concentration of the compound in humans post administration of the compound at dosage Y. In certain embodiments, N-acetylcysteine may be administered multiple times. In certain embodiments, sodium thiosulfate (e.g., at 20 gm/m²) may be administered in combination with another thiol-based compound such as N-acetylcysteine. In certain other embodiments, sodium thiosulfate may be administered alone.

Thiol-based compounds may be administered to a subject in need thereof prior to, concurrent with, or following the administration of chemotherapeutic agents. For instance, thiol-based compounds may be administered to a subject at least or about 4 hours, 3 hours, 2 hours, 1.5 hours, 1 hour, 45 minutes, 30 minutes or 15 minutes before the starting time of the administration of chemotherapeutic agent(s). In certain embodiments, they may be administered concurrent with the administration of chemotherapeutic agent(s). In other words, in these embodiments, thiol-based compounds are administrated at the same time when the administration of chemotherapeutic agent(s) starts. In other embodiments, thiol-based compounds may be administered following the starting time of administration of chemotherapeutic agent(s) (e.g., at least or about 30 minutes, 1 hour, 2 hours, 3 hours, 4 hours, 5 hours, 6 hours, 7 hours or 8 hours after the starting time of administration of chemotherapeutic agents). Alternatively, thiol-based compounds may be administered at least 30 minutes, 1 hour, 2 hours, 3 hours, 4 hours, 5 hours, 6 hours, 7 hours or 8 hours after the completion of administration of chemotherapeutic agents. Generally, these thiol-based compounds are administered for a sufficient period of time so that mucosal injury is prevented or reduced. Such sufficient period of time may be identical to, or different from, the period during which chemotherapeutic agent(s) are administered. In certain embodiments, multiple doses of thiol-based compounds are administered for each administration of a chemotherapeutic agent or a combination of multiple chemotherapeutic agents.

In certain embodiments, an appropriate dosage of a thiol-based compound (e.g., N-acetylcysteine) is combined with a specific timing and/or a particular route to achieve the optimum effect in preventing or reducing mucosal injury. For instance, N-acetylcysteine may be administered to a human being at 150-1400 mg/kg via i.v. at least or about 15 minutes, 30 minutes, 45 minutes or 1 hour prior to the beginning of the administration of a chemotherapeutic agent or a combination of chemotherapeutic agents.

Each thiol-based chemoprotectant agent, such as NAC or STS, can be synthesized by conventional methods and are commercially available as a sterile solution.

### EXAMPLES

### Example 1

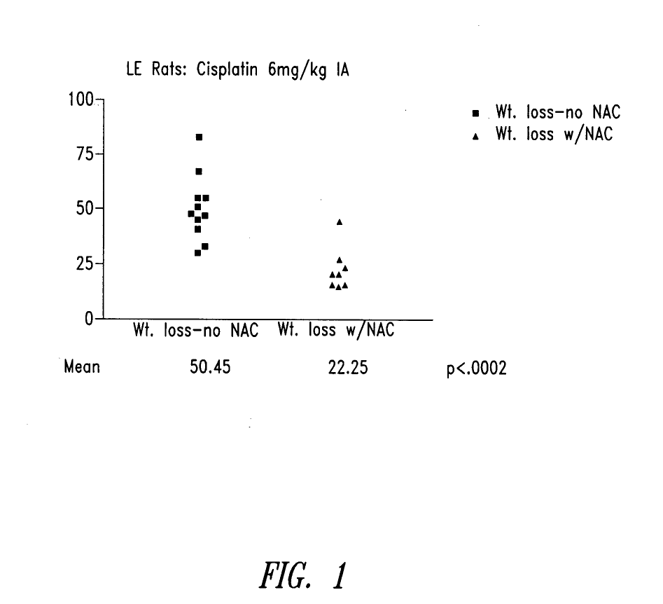

The graph shown as FIG. 1 represents the weight loss in grams in Long Evans rats seven days after administration of Cisplatin and NAC. Six mg/kg of Cisplatin was given intra-arterially either with or without intravenous (i.v.) administration of NAC at a dosage of 400 mg/kg. The NAC was administered about 15 minutes prior to the Cisplatin administration. The group without NAC consisted of 11 members. The NAC-treated group consisted of 8 members. An unpaired t-test showed a significant difference between the groups of p<0.0002.

In the method of Example 1 for Cisplatin, the Long Evans rats were weighed, induced with isoflurane inhalant (2%+1.5% O₂), intubated, placed on a respirator, and prepped for surgery. Isoflurane was then replaced with propofol (800 ug/kg/min i.v.) and a 50% nitrous/50% oxygen mixture. A ventral midline incision was made from mandible to manubrium. The left carotid bifurcation was exposed and freed, and the left external carotid artery was cannulated. NAC was then given intravenously to the treated group and intravenous saline was given to the untreated group. After 15 minutes, the left internal carotid artery was clamped, and Cisplatin (6 mg/kg) was infused through the external carotid catheter. The cannula was removed, and the skin incisions were closed using 4-0 Vicryl in a simple continuous pattern.

Seven days after Cisplatin infusion, the rats were weighed. These body weights were compared to those taken before surgery. The group that did not receive NAC prior to Cisplatin (n=11) had a significantly larger loss of body weight after 7 days when compared to the NAC-treated group (n=8). The group that did not receive NAC experienced greater diarrhea than the NAC treated group. Subjectively, the untreated rats were more depressed, cachexic, and dehydrated than the NAC-treated group. This is correlated with renal failure and/or mucositis in the untreated group.

### Example 2

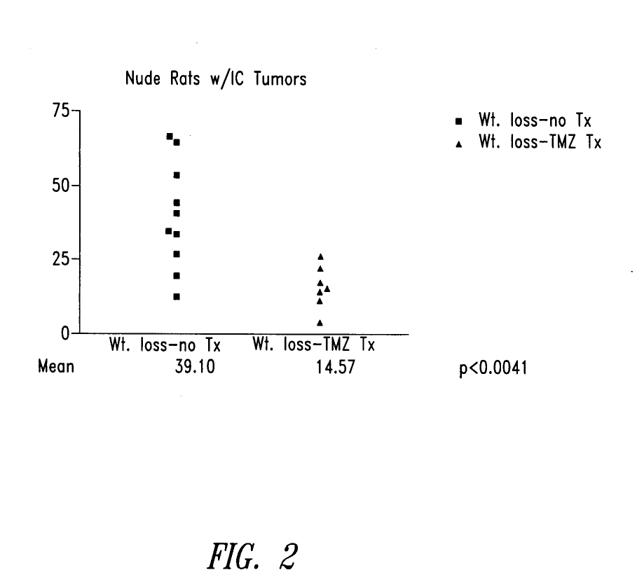

FIG. 2 shows a graph representing the weight loss in grams in nude rats 12 days after intracerebral inoculation with small cell lung cancer cells (SCLC), with and without treatment with Temazolamide (TMZ). TMZ was given at a dosage of 5 mg orally, once daily, on days 6-11. There were 10 members in the untreated group and seven members in the group treated with TMZ. An unpaired t-test showed a significant difference between groups, p<0.0041.

For the intracerebral inoculations, the nude rats were anesthetized with a mixture of intraperitoneal ketamine (60 mg/kg) and diazepam (7.5 mg/kg). The head was shaved and scrubbed with Betadine, a midline incision made, and a 2 mm burr hole using stereotaxic coordinates was made. A 27 g needle was used for inoculation with a 100 ul Hamilton syringe, placed 0.65 mm ventral to the surface, deep in the right caudate putamen. Inoculants used were LX-1 SCLC. The needle was withdrawn and the incision closed with wound clips. One group of rats received no treatment, while the other group received oral 5 mg doses of TMZ, an alkylating agent, once daily on days 6-11 after inoculation, delivered through a feeding tube while under isoflurane anesthesia. On day 12, all rats were weighed, sacrificed, and the brains removed and fixed in formalin for subsequent tumor-volume analysis.

The group that did not receive TMZ had a significantly higher loss of body weight than did the group that was treated with TMZ. It is believed that TMZ effects the size of any lesions in the brain. The TMZ may decrease the size of any lesions in the brain; those animals experiencing more constant or improved appetite than animals with larger tumors which experienced appetite suppression. Also, three rats without TMZ did not survive to day 12 and were not included in the study, while all of the TMZ rats survived to day 12. Subjectively, the TMZ-treated rats were more active and responsive at day 12 than the untreated group.

### Example 3

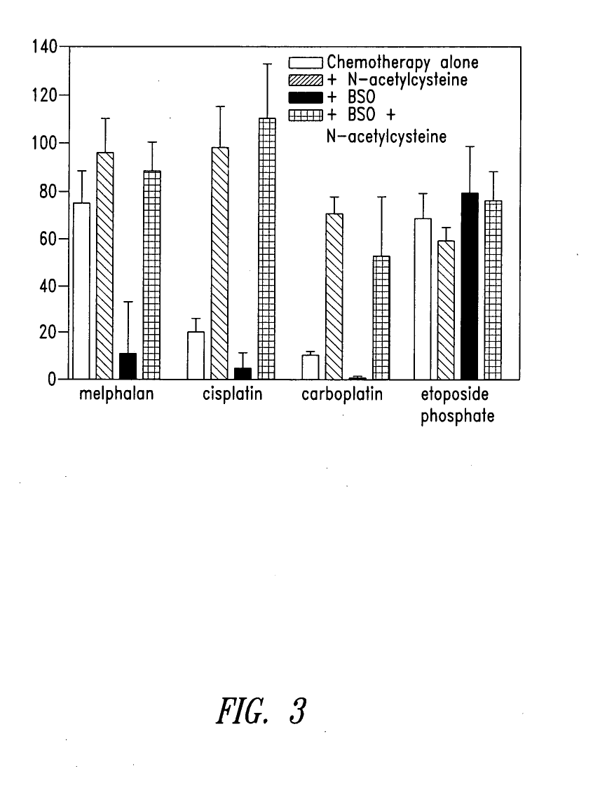

As shown in FIG. 3, the anti-mucosal injury effect seen in the LX-1 SCLC cells was further evaluated by testing in the GM294 human fibroblast cell strain.

The fibroblast cells were either preincubated or not preincubated with 100 μM BSO for about 18-24 hours prior to addition of chemotherapy, and rescue consisted of 1000-2000 μg/ml of a thiol anti-mucosal injury agent added immediately after chemotherapy. As shown in FIG. 3, NAC had a protective effect on fibroblast cells whether treated or pretreated with BSO and a chemotherapeutic agent such as melphalan, cisplatin, carboplatin or etoposide phosphate or if not pretreated with BSO and administered NAC with chemotherapeutic agent. As fibroblasts are precursor cells to epithelia cells, protection of fibroblast cells demonstrates protection of subsequent epithelia cells. Protection of epithelia cells allows for the prevention and treatment of mucositis of these tissues.

### Example 4

Normal human gastric cells (NHGC) express high levels of the LewisY antigen recognized by the BR96 monoclonal antibody. BR-96 and BR-96-Dox toxicity was examined in NHGC to determine if toxicity could be protected against with thiol chemoprotectants.

Cytotoxicity is determined as the proportion of live cells, in comparison to untreated controls, using the WST-1 colorometric assay available from Roche Diagnostics, Indianapolis, Ind.

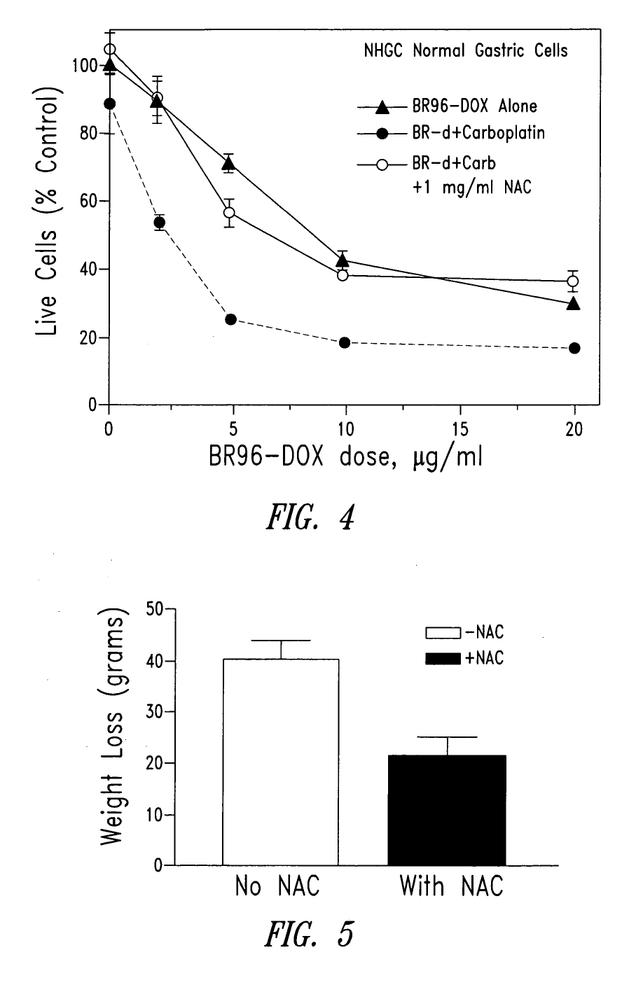

As shown in FIG. 4, in the first test, the drug and chemoprotectant were co-incubated for 2-3 days. No chemoprotection was seen against BR96-dox toxicity with STS, GSH ethyl ester, or d-Methionine. Low dosages, about 0.2 to about 0.5 mg/ml, of NAC were protective against alkylators in LX-1 cells. NAC was 10-90% chemoprotective of NHGC at high dosages, such as from about 1-5 mg/ml.

In a second test, a 4 hour pulsed treatment with BR-96-dox and NAC was 0-20% chemoprotective.

In a third test, a 48 hour co-incubation of drug and chemoprotectant in LX-1 cells did not show a chemoprotective effect.

As there was shown a chemoprotective effect in the first and second above tests with NHGC, one can conclude that NAC is chemoprotective of gastric cells, the cells that line the gastro-intestinal track. FIG. 4 shows the chemoprotective effect of NAC on NHGC, the cells involved in mucosal injury.

### Example 5

FIG. 5 shows the results of the administration of NAC and cisplatin in Long Evans rats on weight loss. Before the study began, the rats were weighed to establish a baseline. Long Evans rats were treated with cisplatin 6 mg/kg delivered to the aorta via a right external carotid artery cannula 15 minutes after intravenous infusion of saline (n=8) or NAC 400 mg/kg (n=8). This procedure was preformed by the rats being induced with isoflurane inhalant (2%+1.5% oxygen), intubated, placed on a respirator and prepped for surgery. Isoflurane was then replaced with propofol (800 μg/kg/min i.v.) and a 50% nitrous/50% oxygen mixture. A ventral midline incision was made from mandible to manubrium. The right carotid bifurcation was exposed and freed, and the right external carotid artery was cannulated. NAC (400 mg/kg) was then given to the treated group (n=8) and i.v. saline was given to the untreated group (n=8). After 15 minutes, the right internal carotid artery was clamped and cisplatin (6 mg/kg) was rapidly infused through the external carotid catheter. The cannula was removed and the skin incisions were closed using 4-0 Vicryl in a simple continuous pattern. Health and well being were monitored daily post-surgery. After seven days, the animals were weighed and this value compared to the baseline.

As shown in FIG. 5, after 7 days, the animals treated with NAC experienced less weight loss than the control. This data shows that treatment with NAC can prevent weight loss and is protective against mucosal injury, including gastrointestinal toxicity.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for preventing or ameliorating chemotherapeutic agent-induced mucosal injury, comprising administering to a patient in need thereof an effective amount of a thiol-based compound or composition prior to, concurrently with, or following the administration of a chemotherapeutic agent or chemotherapeutic agents.

2. The method according to claim 1 wherein the thiol-based compound is administered orally.

3. The method according to claim 1 wherein the thiol-based compound is administered intravenously.

4. The method according to claim 1 wherein the thiol-based compound is administered intra-arterially.

5. The method according to claim 1 wherein the thiol-based compound is administered prior to the administration of the chemotherapeutic agent or at least one of the chemotherapeutic agents.

6. The method according to claim 1 wherein the thiol-based compound is administered concurrently with the administration of the chemotherapeutic agent or at least one of the chemotherapeutic agents.

7. The method according to claim 1 wherein the thiol-based compound is administered following the administration of the chemotherapeutic agent or at least one of the chemotherapeutic agents.

8. The method according to claim 7 wherein the thiol-based compound is administered at least or about 60 minutes prior to the beginning of the administration of the chemotherapeutic agent or at least one of the chemotherapeutic agents.

9. The method according to claim 7 wherein the thiol-based compound is administered at least or about 30 minutes prior to the beginning of the administration of the chemotherapeutic agent or at least one of the chemotherapeutic agents.

10. The method according to claim 7 wherein the thiol-based compound is administered at least or about 15 minutes prior to the beginning of the administration of the chemotherapeutic agent or at least one of the chemotherapeutic agents.

11. The method according to claim 1 wherein the thiol-based compound is selected from the group consisting of sodium thiosulfate, N-acetylcysteine, glutathione ethyl ester, glutathione, D-methionine, cysteramine, cystamine, aminopropylmethylisothiourea, and combinations thereof.

12. The method according to claim 1 wherein the thiol-based compound is sodium thiosulfate.

13. The method according to claim 1 wherein the thiol-based compound is N-acetylcysteine.

14. The method according to claim 1 wherein the thiol-based composition comprises sodium thiosulfate and N-acetylcysteine.

15. The method according to claim 1 wherein the chemotherapeutic agent is an alkylating agent.

16. The method according to claim 15 wherein the alkylating agent is a platinum-containing alkylating agent.

17. The method according to claim 16 wherein the platinum-containing alkylating agent is selected from the group consisting of cisplatin, carboplatin, and oxyplatin.

18. The method according to claim 1 wherein the chemotherapeutic agent is carboplatin or BR96-dox.

19. The method according to claim 1 wherein the patient in need thereof is a human.

20. The method according to claim 19 wherein the thiol-based compound is N-acetylcysteine.

21. The method according to claim 24 wherein the chemotherapeutic agent is carboplatin.

22. The method according to claim 20 wherein N-acetylcysteine is administered at a dosage 150-1,400 mg/kg.

23. The method according to claim 22 wherein N-acetylcysteine is administered intravenously.

24. The method according to claim 23 wherein N-acetylcysteine is administered about 15-30 minutes prior to the beginning of the administration of the chemotherapeutic agent or at least one of the chemotherapeutic agents.
